(19) United States
(12) Patent Application Publication
(10) Pub. No.: US 2024/0197829 A1
(43) Pub. Date: Jun. 20, 2024

(54) TREATMENT OF NEUROPATHIC CORNEAL PAIN WITH NGF

(71) Applicants: DOMPÉ FARMACEUTICI S.P.A, MILANO (IT); TUFTS MEDICAL CENTER, INC., Boston, MA (US)

(72) Inventors: Pedram HAMRAH, Wellesley, MA (US); Flavio MANTELLI, Rome (IT)

(21) Appl. No.: 18/554,511

(22) PCT Filed: Apr. 13, 2022
(86) PCT No.: PCT/US2022/024519

§ 371 Date: Oct. 9, 2023

Related U.S. Application Data
(60) Provisional application No. 63/174,163, filed on Apr. 13, 2021.

(51) Int. Cl. A61K 38/18; A61P 25/04; A61P 27/02

(52) U.S. Cl. CPC A61K 38/185; A61P 25/04; A61P 27/02

(57) ABSTRACT

The present invention relates to methods of treating neuropathic corneal pain (NCP) comprising administration of nerve growth factor (NGF).

### FIELD OF THE INVENTION

The present invention relates to methods of treating neuropathic corneal pain (NCP) comprising administration of nerve growth factor (NGF).

### BACKGROUND OF THE INVENTION

The nerve growth factor (NGF) is a member of the family of evolutionarily well-conserved neurotrophin growth factors that are required for the development and survival of specific neuronal populations, which also includes brain-derived neurotrophic factor (BDNF), neurotrophin-3 (NT3) and NT4/5. The NGF sequence is well preserved among different species, with 90% homology between murine and human NGF.

Recombinant human nerve growth factor (rhNGF) has been approved by the U.S. Food and Drug Administration (FDA) and the European Medicines Agency (EMA) for use in the treatment of neurotrophic keratitis, a rare degenerative ocular disease of the cornea characterized by reduction or loss of corneal sensitivity that can be asymptomatic or present with red-eye and, during the early stages of the disease, a minor decrease in visual acuity. It eventually leads to loss of vision. Recombinant human nerve growth factor (rhNGF) has also been shown to be safe and effective in improving symptoms and signs of dry eye disease (DED) in a human clinical trial (Sacchetti, et al., Br. J. Ophthalmol., 2020, 104, 127-135).

Dry eye disease (DED) is a multifactorial disease of the ocular surface characterized by a loss of homeostasis of the tear film, and accompanied by ocular symptoms, in which tear film instability and hyperosmolarity, ocular surface inflammation and damage, and neurosensory abnormalities play etiological roles (Craig, et al., Ocul Surf., 2017, 15, 276-283).

Neuropathic corneal pain (NCP), also known as neuropathic ocular pain, corneal neuralgia, keratoneuralgia, neuropathic like pain, chronic ocular surface pain, results from a complex interplay of various central and peripheral mechanisms and causes perceptions such as burning, stinging, eye-ache, and pain (Goyal and Hamrah, Seminars in Ophthalmology, 2016, 31, 59-70).

The International Association for the Study of Pain defines neuropathic pain as “pain initiated or caused by a primary lesion or dysfunction of the nervous system”. The diagnosis of neuropathic pain thus requires confirmation of injury or disease affecting somatosensory pathways of peripheral and/or central nervous systems (CNS).

In patients with NCP, the Proparacaine Challenge Test is used to distinguish between peripheral and central pain. Treatment with a topical anesthesia drop of 0.5% proparacaine hydrochloride will attenuate peripheral but not central pain (Goyal and Hamrah).

More recently, the following subtypes of NCP have been identified: patients may fully (peripheral NCP; ˜25%) or partially (mixed NCP; ˜50%) respond to anesthetic drops or patients may not respond or worsen with anesthetic drops (unpublished data).

In patients suffering from ocular discomfort/pain, the discomfort/pain may be characterized as the following three types: nociceptive pain, inflammatory pain, and neuropathic pain. These different types of pain may also co-exist.

Inflammation may be detected clinically (by redness on slit-lamp biomicroscopy); however, in many cases inflammation may only be detected sub-clinically (e.g., by in vivo confocal microscopy by the presence of dendritiform cells).

Dendritiform cell (DC) density in normal subjects is 25.9±3.9 cells/mm². A DC density which is two standard deviations higher than normal (i.e., above 75 cells/mm²) indicates the presence of severe inflammation.

Recent data has shown that in conventional dry eye disease (n=300 eyes), DC density is 93.4±6.3 (Aggarwal, et al., Ocul Surf., 2021, 19, 183-189).

Patients with neuropathic corneal pain (NCP) have a mean DC density of 71.89±16.91 (Moein, et al., Ocul Surf., 2020, 18, 651-656).

The level of inflammation in NCP patients may be highly variable, with Inflammation that may be absent, mildly increased (less than 2 SD above normal levels), or significantly increased (more than 2 SD above normal levels).

### SUMMARY OF THE INVENTION

The present inventors have found that, in patients suffering from neuropathic corneal pain (NCP) who respond to treatment with anesthetic drops, NGF is effective in treating NCP. Moreover, this result has been confirmed in a murine mouse model for NCP which does not induce dry eye disease (DED).

Thus, the present invention relates to a method of treating neuropathic corneal pain (NCP) in a subject in need thereof, comprising administration of a composition comprising NGF.

The present invention also relates to NGF for use in the treatment of neuropathic corneal pain (NCP) in a subject in need thereof.

A further aspect of the invention relates to such a method comprising administration of a composition comprising NGF, preferably rhNGF.

A further aspect of the invention relates to NGF for use as defined above, wherein said NGF is rhNGF.

A further aspect of the invention relates to a method or to NGF for use as defined above, wherein the subject is a patient suffering from NCP and wherein the patient is responsive to topical anesthesia treatment.

A further aspect of the invention relates to a method or to NGF for use as defined above, wherein the subject is a patient suffering from peripheral or mixed NCP.

A further aspect of the invention relates to a method or to NGF for use as defined above, wherein the subject is a patient suffering from NCP with mild inflammation.

A further aspect of the invention relates to a method or to NGF for use as defined above, wherein said patient has a Dendritiform Cell Density (cells/mm²) lower than 75, preferably lower than 70.

A further aspect of the invention relates to a method or to NGF for use as defined above, wherein said patient does not have dry eye disease (DED).

A further aspect of the invention relates to a method or to NGF for use as defined above, wherein said patient does not have neurotrophic keratitis (NK).

A further aspect of the invention relates to a method or to NGF for use as defined above, wherein a composition comprising NGF is topically applied to an ocular surface of the subject.

### BRIEF DESCRIPTION OF THE DRAWINGS

FIG. 1 shows the response to [5M] Saline challenge at 3 days post surgery (dps) for the murine model of NCP, demonstrating that there is an enhanced response in those animals that underwent ciliary nerve ligation compared to sham procedures.

FIG. 2 shows corneal fluorescein staining according to the NEI scale (0-15) for the murine model of NCP, with higher scores indicating a worsening defect in the corneal epithelium. The data shown demonstrate that there are no significant differences in CFS between groups at any time point (p>0.05), and this importantly indicates that the ligation model is inducing a pure NCP phenotype, and not a dry eye disease phenotype.

FIG. 3 shows corneal sensitivity in the murine model of NCP, as measured by Cochet-Bonnet esthesiometry, which is an objective method of determining corneal nerve sensitivity by use of a monofilament at different lengths, thereby applying different pressures to the cornea.

FIG. 4 shows the response to [5M] saline challenge throughout the course of the study in the murine model of NCP. There is an observable decrease in the response to [5M] saline in the NGF treated group for both doses at 7, 10, and 14 dps (p<0.05). By 14 dps, the NGF 4×/day group is approaching a return to the baseline response observed prior to the induction of NCP and the NGF 6×/day group has a complete return to baseline.

FIG. 5 shows the response to cold saline challenge in the murine model of NCP. The responses were variable, and interestingly, there was a significant difference between the NGF 4λ/day and vehicle groups at days 7, 10 and 14.

FIG. 6 shows the response to L-menthol challenge in the murine model of NCP. These responses were also variable. There was a significant difference between the groups at early time points but this was lost by day 14.

### DETAILED DESCRIPTION

The present invention is based on the discovery that NGF is effective in treating neuropathic corneal pain (NCP) in a specific and well identified subset of patients suffering from NCP.

In particular, NGF is effective in treating patients suffering from NCP who respond to treatment with a topical anesthetic (e.g., proparacaine) and who have mild inflammation as seen by IVCM. The use of NGF in this patient population results in improvements in decreased pain levels and improvements in inflammation.

Accordingly, an object of the invention is a method of treating neuropathic corneal pain (NCP) in a subject in need thereof, comprising administration of a composition comprising NGF.

A further object of the invention is NGF for use in the treatment of neuropathic corneal pain (NCP) in a subject in need thereof.

Said subject belongs to the patient population for whom said NGF treatment is effective, as described below.

According to the present invention, the patient population for whom NGF treatment is effective may be defined as patients suffering from NCP who respond to treatment with a topical anesthetic, preferably proparacaine and/or who have a mild eye inflammation.

The expression “respond to treatment with topical anesthetic” according to the present invention refers to a partial or complete relief of pain as a result of topical application to the eye of patients of anesthetic drops for topical application, preferably wherein the anesthetic is proparacaine, more preferably proparacaine hydrochloride, more preferably proparacaine hydrochloride at a dosage of 0.5%.

Preferably, said patients who have mild inflammation are those having a Dendritiform Cell Density (cells/mm²) lower than 75, preferably lower than 70.

The above patient population may be further defined as patients suffering from peripheral NCP or mixed NCP (i.e., patients who respond fully, with complete relieve of pain, or partially, with partial relief of pain, to anesthetic drops, respectively).

In addition to the above-mentioned characteristics, preferably, the patient population according to the present invention may be further defined as patients who do not suffer from dry eye disease (DED).

In addition to the above-mentioned characteristics, preferably, the patient population according to the present invention may be further defined as patients who do not suffer from neurotrophic keratitis (NK).

As used herein nerve growth factor (NGF) includes murine NGF (mNGF) and recombinant human nerve growth factor (rhNGF), including cenegermin-bkbj.

Preferably said NGF is rhNGF.

mNGF for use according to the invention may be manufactured in E. Coli, for example according to the process described in WO2000/022119 and WO2013/092776, using an expression vector incorporating the sequence of the proNGF mutant SP174-101 (SEQ ID NO: 5 of WO2013/092776).

Preferably, according to the invention the NGF is administered in the form of a pharmaceutical composition for ophthalmic use.

Preferably, said NGF is administered topically to an ocular surface of the subject.

The exact dose and regimen for administration of NGF in the treatment or the prevention of the above pathologies will depend upon many factors, such as for instance the route of administration and the severity of the disease of the individual receiving treatment.

Said pharmaceutical composition comprises the above-described NGF and one or more ophthalmologically acceptable excipients.

An “ophthalmologically acceptable excipient” is an inert excipient which allows delivery of a medicament to the eye and/or eyelids, to treat an ocular disease or condition without deleterious effects on the eye.

According to an embodiment, said ophthalmic composition may be a liquid, eye drop composition for topical administration to the anterior segment of the eye.

Said liquid composition may be in form of a solution, emulsion, or suspension. Said liquid composition may include micelles. In one embodiment, the liquid composition is an aqueous composition.

Preferably, the liquid composition is an aqueous eye drop composition.

According to an alternative preferred embodiment, said ophthalmic formulation is a semi-solid ophthalmic formulation, preferably a cream, ointment or gel.

According to yet another preferred embodiment, said ophthalmic formulation is a solid ophthalmic formulation for the extemporaneous preparation of a liquid or semi-solid ophthalmic formulation, as described above, by addition of a diluent before administration. Preferably, said solid ophthalmic formulation is in form of a powder, more preferably it is in the form of a lyophilized powder.

Preferably, said liquid or semi-solid composition comprises ophthalmologically acceptable excipients selected from ophthalmologically acceptable viscosity enhancers, penetration enhancers, buffering agents, osmolarity regulators, preservatives and surfactants.

Viscosity enhancers have the function to increase viscosity of the composition and to improve its retention in the conjunctival sac and are preferably selected from cellulose derivatives, preferably hydroxymethylcellulose, hydroxyethylcellulose, hydroxypropylmethylcellulose, methylcellulose; polyvinylpyrrolidone and gelling agents, preferably gellan, xanthan gum and carbopol-974.

Penetration enhancers have the function of enhancing drug permeability across ocular membranes and are preferably selected from cyclodextrins, chelating agent, crown ethers, bile acids and bile salts.

Buffering agents have the function of providing and maintaining the correct pH of the formulation to be compatible for use in the eye, preferably at a pH comprised between 6 and 8. The preferred buffer is phosphate buffer, but other buffers capable of maintaining the pH within the desired range, especially buffers suitable for ophthalmic use, are also included.

Osmolarity regulators are salts able to make the liquid composition isotonic with ocular fluids. The preferred salt is sodium chloride (NaCl) but other biologically acceptable salts may be used, such as for instance potassium chloride (KCl), calcium chloride (CaCl₂)) and magnesium chloride (MgCl₂) and their admixtures.

Preservatives inhibit microbial activity. Suitable preservatives include for instance quaternary ammonium compounds such as benzalkonium chloride, cetyltrimethylammonium bromide and cetylpyridinium chloride.

Surfactants have the function of making the composition stable and of reducing or preventing NGF adsorption to various surfaces of the container and are preferably selected from polysorbates such as Tweeny 80, poloxamers such as Pluronics F68 or proteins such as serum albumin.

Said liquid, eye drop composition can be part of a kit comprising the composition, a container for holding the composition and a drop dispenser.

The NGF aqueous composition according to the invention may comprise a sufficient amount of biologically acceptable salts to provide the correct fluid tonicity and to maintain the NGF in solution.

Other additives commonly used in pharmaceutical aqueous compositions and known to the technical expert, such as sugars, sugar alcohols, aminoacids, cellulose-derivatives, polyethylene glycols, may be present in the NGF aqueous composition.

The NGF aqueous composition comprises water in an amount sufficient to achieve the appropriate concentration of composition components.

The liquid composition comprises NGF in therapeutically effective concentrations.

Preferably, in the liquid composition, NGF is present at concentrations ranging from about 0.0001% to about 0.5% w/v, more preferably from about 0.001% to about 0.1% w/v, most preferably of about 0.002% w/v of the aqueous composition.

Preferably said NGF is administered between 3 and 6 times a day for a period between 4 and 12 weeks. The administration schedule will be determined by the physician based on the dosage of the NGF formulation and the severity of the condition of the patient.

Some aspects of the invention will be better described in the following:

In a first aspect, it is provided NGF for use in the treatment of neuropathic corneal pain (NCP) in a subject in need thereof.

In a second aspect, according to the preceding aspect, said NGF for use is selected from murine NGF (mNGF) and recombinant human nerve growth factor (rhNGF), preferably it is recombinant human nerve growth factor (rhNGF).

In a third aspect, according to any of the preceding aspects, said subject is a patient suffering from Neuropathic Corneal Pain and who responds to treatment with a topical anesthetic and/or who has a mild eye inflammation.

In a fourth aspect, according to the third aspect, said subject is a patient having a partial or complete relief of pain as a result of topical application to the eye of anesthetic drops for topical application.

In a fifth aspect, according to any of the third and fourth aspect, said anesthetic is proparacaine, more preferably proparacaine hydrochloride, more preferably proparacaine hydrochloride at a dosage of 0.5%.

In a sixth aspect, according to any of the preceding aspects, said patient have a Dendritiform Cell Density (cells/mm²) lower than 75, preferably lower than 70.

In a seventh aspect, according to any of the preceding aspects, said subject does not suffer from dry eye disease (DED).

In an eighth aspect, according to any of the preceding aspects, said subject does not suffer from neurotrophic keratitis (NK).

In a nineth aspect, according to any of the preceding aspects, said NGF for use is administered topically to an ocular surface of the subject.

In a tenth aspect, according to any of the preceding aspects, said NGF is administered in the form of a pharmaceutical composition for ophthalmic use.

In an eleventh aspect, it is provided a pharmaceutical composition for use in the treatment of neuropathic corneal pain (NCP) in a subject in need thereof, as defined in any of the above second to eight aspects.

In a twelfth aspect, according to any of the tenth and eleventh aspects, said pharmaceutical composition is a liquid composition for topical administration to the anterior segment of the eye.

In a thirteenth aspect, according to the twelfth aspect, said liquid composition is an aqueous composition, preferably an aqueous eye drop composition.

In a fourteenth aspect, according to any of the tenth and eleventh aspects, said pharmaceutical composition is semi-solid ophthalmic formulation, preferably a cream, ointment or gel.

In a fifteenth aspect, according to any of the tenth and eleventh aspects, said pharmaceutical composition is a solid ophthalmic formulation for the extemporaneous preparation of a liquid or semi-solid ophthalmic formulation according to the twelfth or fourteenth aspect, respectively.

### EXAMPLES

The following examples illustrate the invention without limiting its scope.

### Example 1—Retrospective Case Study of Patients with Neuropathic Corneal Pain (NCP)

A retrospective case series of 20 patients with neuropathic corneal pain, seen at the Cornea Service of the New England Eye Center, Tufts Medical Center, Boston, MA, between December 2018 and January 2021, was performed. Patients were diagnosed with NCP when symptoms were out of proportion to signs, patients' pain did not respond to extensive prior therapies, symptoms did not resolve with anesthetic drops after 90 seconds, or patients demonstrated microneuromas on IVCM. In cases with complete resolution of pain to anesthetic drops, lack of pain response to extensive prior therapies, symptoms out of proportion to signs, decreased cornea sensation, and presence of microneuromas were used for the diagnosis of NCP. A thorough chart review was performed, and clinical parameters at two time points were recorded, before treatment with recombinant nerve growth factor (Oxervate™). Patient used rhNGF (Oxervate™ 6×/day for 8 weeks). After starting rhNGF therapy, the patients were followed, and severity of patient-reported pain was recorded and IVCM was conducted before and after completion of 8 week therapy.

The study was approved by the Institutional Board Review of Tufts Medical Center/Tufts University Health Sciences. The protocol conformed to the Declaration of Helsinki and adhered to the Health Insurance Portability and Accountability Act (HIPAA).

Data extracted from the medical records included patient demographics, clinical findings, including vital dye staining by Oxford Scale, tear metrics, and symptom questionnaires, including ocular surface disease Index (OSDI), as well as the ocular pain assessment survey (OPAS). Moreover, proparacaine challenge test (PCT) results from the same visit were recorded. For the PCT, patients were asked to report their pain relief based on visual analogue scale after 90 seconds of installation of 0.5% proparacaine hydrochloride eye drops (Alcaine; Novartis Ophthalmics, East Hanover, NJ).

Laser IVCM (Heidelberg Retina Tomograph 3 with the Rostock Cornea Module, Heidelberg Engineering GmbH, Heidelberg, Germany) was conducted routinely on all patients presenting with pain and discomfort for confirmation of corneal subbasal nerve alterations, as previously described (Cruzat, et al., IVOS, 2011, 52, 5136-5143). IVCM was obtained from all patients and compared to age- and sex-matched reference controls from a prospectively enrolled normative database. Images were obtained with the help of a 63× objective immersion lens with a numerical aperture of 0.9 (Olympus, Tokyo, Japan). This microscope uses a 670-nm red wavelength diode laser source producing images representing a coronal section of the cornea of 400×400 μm (horizontal×vertical). Digital images were recorded at of 30 frames/s. Adjacent images are separated by 1 μm, with a lateral resolution of 1 μm/pixel. To perform this procedure, both eyes were topically anesthetized using 0.5% proparacaine hydrochloride (Alcaine; Novartis Ophthalmics). This was followed by administration of a drop of hydroxypropyl methylcellulose 2.5% (GenTeal gel, Alcon, Fort Worth, TX) to improve the optical coupling with the cornea module of the microscope. The cornea module was mounted with a disposable, sterile polymethylmethacrylate cap (Tomo-Cap: Heidelberg Engineering GmbH), filled with a layer of hydroxypropyl methylcellulose 2.5% (GenTeal gel; Alcon), gel was also applied to the surface of the cap. The equipment is manually advanced until the gel on the cap comes in contact with the surface of the central cornea. Fifty to hundred images of the corneal subbasal layer were obtained via six to eight sequence, and a masked observer (P.H.) selected the three most representative images determined as best focused, single layer, minimum folds and good contrast of the subbasal nerve plexus. One masked observer (P.H.) analyzed IVCM images for density of dendritiform cells (DCs).

As can be seen by the data shown in Table 1, patients who responded to proparacaine and had mild inflammation as seen by IVCM, responded to Oxervate™ treatment and had significant improvement (i.e., decreased pain levels). Patients who did not respond to proparacaine or had severe inflammation did not show decreased pain levels in response to Oxervate™ treatment. Further, the patients with no, mild, or moderate inflammation also showed improvements with respect to inflammation.

TABLE 1

| | Responder | Non-Responder |
|---|---|---|
| | Cases | Cases |
| | (n = 13) | (n = 7) |
| Parameter | (mean ± SD) | (mean ± SD) |
| Age (years) | 61.7 ± 11.6 (44-73) | 57.9 ± 8.6 (41-72) |
| Gender (female/male) | 7/6 | 4/3 |
| Schirmer's score Il (mm) | 7.00 ± 4.08 | 8.3 ± 2.13 |
| Corneal fluorescein | 3.43 ± 0.79 | 3.13 ± 0.54 |
| staining (0-5 Oxford | | |
| scale) | | |
| OSDI | 72.26 ± 6.81 | 78.45 ± 5.63 |
| Pain Visual Analogue | 6.24 ± 1.65 | 7.34 ± 2.35 |
| Scale | | |
| Pre-Treatment | | |
| Pain Visual Analogue | 1.66 ± 2.24 | 5.45 ± 1.67 |
| Scale | | |
| Post-Treatment | | |
| Response to | 13 | 3 |
| Proparacaine by more | | |
| than 50% on VAS | | |
| Dendritiform Cell Density | 53.2 ± 13.4 | 131.3 ± 39.5 |
| (cells/mm²) on In Vivo | | |
| Confocal Microscopy | | |
| (mean ± SD) Pre- | | |
| Treatment | | |
| Dendritiform Cell Density | 31.2 ± 9.2 | 110.3 ± 41.1 |
| (cells/mm²) on In Vivo | | |
| Confocal Microscopy | | |
| (mean ± SD) Post- | | |
| Treatment | | |

### Example 2—Evaluation of Topical NGF in a Murine Model of Neuropathic Corneal Pain

This study was performed to investigate the utility of topical NGF drops (0.02 mg/mL) in the treatment of Neuropathic Corneal Pain (NCP), using a murine model of NCP induced by ciliary nerve ligation.

### Confirmation of Ciliary Nerve Ligation and Induction of NCP

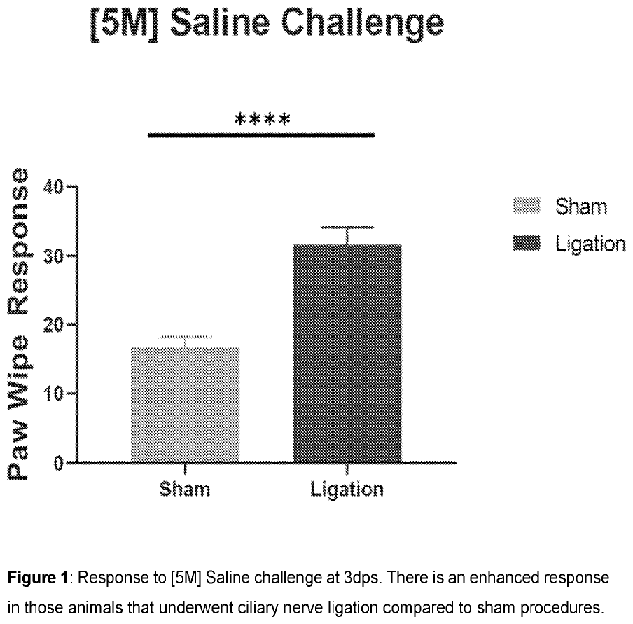

A baseline response to [5M] saline was taken prior to the ligation or sham surgical procedure. At 3 days post surgery (dps), animals were again challenged with [5M] saline. The results, which are shown in FIG. 1, confirm that ligation is successful as judged by the [5M] saline challenge, as those animals undergoing the ligation procedure had an enhanced response (p<0.0001), indicative of an increase in pain.

### Study Design

At Day 3, animals that underwent ligation were distributed into two groups—one group receiving recombinant NGF (0.02 mg/mL) and the other receiving vehicle drops, 6 times per day. Mice were followed up until 14 dps, with outcome measures of corneal fluorescein staining, Cochet-Bonnet esthesiometry, [5M] saline challenge, cold saline challenge, and L-menthol challenge occurring at 7, 10, and 14 dps. At 14 dps, corneas and trigeminal ganglions were collected for further analyses.

### Clinical Outcomes

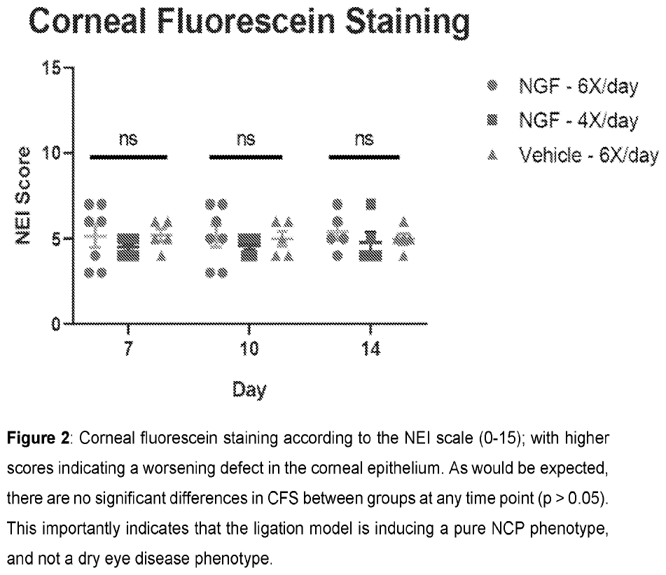

Corneal Fluorescein Staining: Mice were briefly anesthetized with ketamine/xylazine and prepared for examination of the ocular surface by slit-lamp microscopy. A drop of fluorescein was instilled into the ligated eye, with manual blinking to cover the entirety of the ocular surface. Staining was allowed to develop for at least one minute, at which point the ocular surface was assessed for staining and scored by the National Eye Institute scale (0-15). This is a necessary quality control measure to ensure that ligated animals are not developing dry eye disease (NEI score >10), which could confound results. As shown in FIG. 2, there was only slight staining in animals across all groups and time points, this implies that the ligation approach is not inducing dry eye disease, but rather a ‘pure’ neuropathic corneal pain phenotype. Additionally, these staining scores are in line with what may be expected from a naïve mouse (slight staining).

Corneal Sensation: A Cochet-Bonnet esthesiometer was used to confirm that corneal sensation was intact in all animals. This approach uses a fine nylon filament applied to the central cornea. Enough pressure is applied to result in a slight bending of the filament. Based on the length of the filament it is possible to determine the amount of pressure applied. If corneal sensation is intact, the outcome is a blink reflex by the animal.

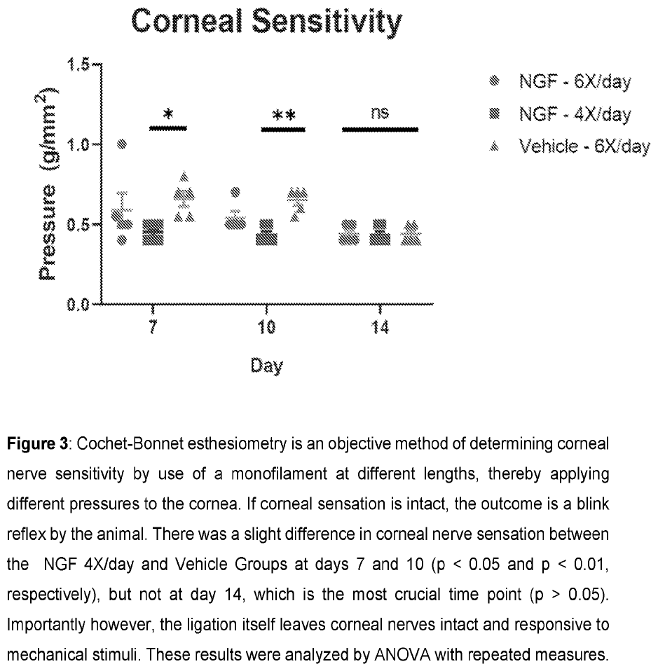

For these assessments, an individual mouse had to have a positive response (blink) in 2/3 attempts, otherwise the length of the filament would be decreased at 5 mm intervals for reassessment (a shorter filament length implies a greater applied pressure). As shown in FIG. 3, there was a slight difference in corneal nerve sensation between the NGF 4×/day and Vehicle Groups at days 7 and 10 (p<0.05 and p<0.01, respectively), but not at day 14, which is the most crucial time point (p>0.05). These results were analyzed by ANOVA with repeated measures.

Importantly, however, the corneal sensation was intact at essentially comparable levels across all groups. This indicates that ligation does not destroy the ciliary nerves, in which case the blink reflex would be completely absent. Instead, the nerves are preserved but dysfunctional.

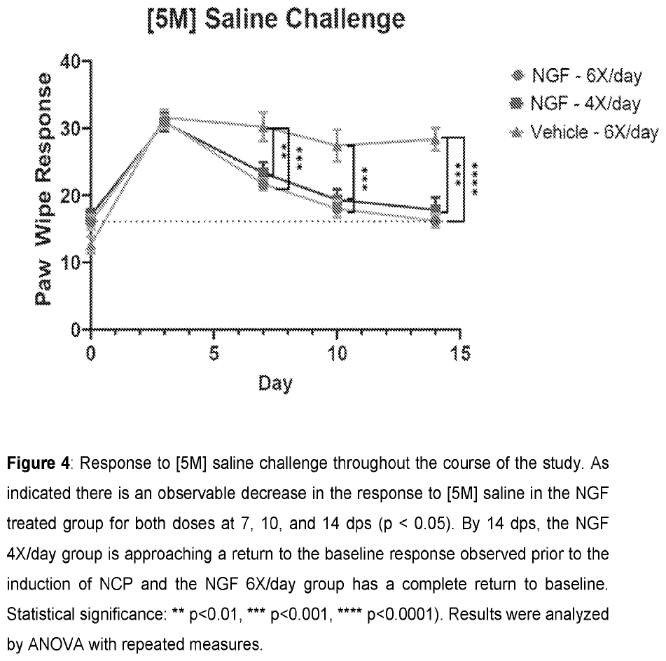

Response to Hyperosmolar Saline: Having confirmed that ligation: (1) was successful, (2) did not result in dry eye disease as assessed by corneal fluorescein staining, and (3) left corneal sensation intact, alterations in pain outcomes were investigated. Hyperosmolar saline solution [5M] was instilled to the ocular surface (10 μL) on the ligated side, within the immediate 30 second interval the number of paw wipes was counted. The number of paw wipes was used as a measure of pain, in that the greater the number of paw wipes performed by an animal then the greater the pain experienced by that animal. The results are shown in FIG. 4.

As can be seen in FIG. 4, there was a similar baseline response between groups at Day 0, as well as at Day 3 post-ligation which was taken before treatment was initiated. In the follow-up period, there was a striking decrease in the number of paw wipes (and therefore pain) in the NGF treated group at both the 6× and 4×/day doses. Most impressively, there was a return to baseline response in the NGF 6×/day group. Additionally, the responses were stable in the vehicle treated group.

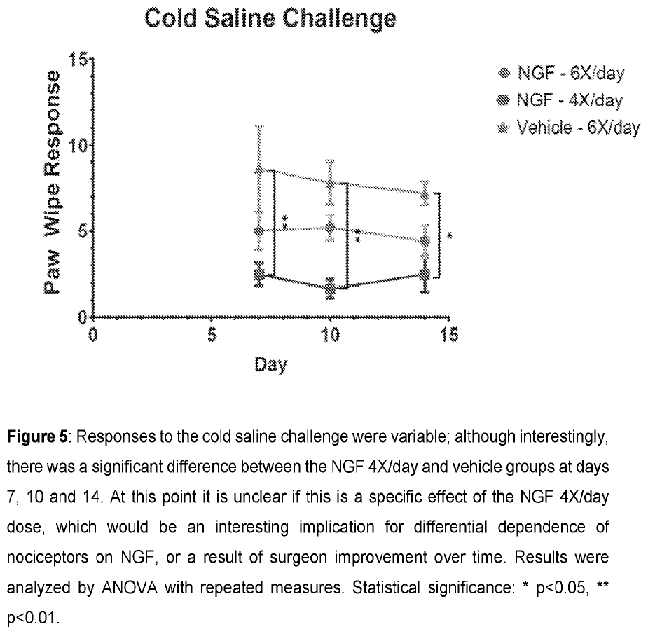

Response to Cold Saline: Cold saline was instilled to the ocular surface (10 μL) on the ligated side, within the immediate 30 second interval the number of paw wipes was counted. The number of paw wipes is used as a measure of pain, in that the greater the number of paw wipes performed by an animal then the greater the pain experienced by that animal. Results of this assessment are shown in FIG. 5. As indicated, there is a statistically significant difference between the NGF 4×/day group compared to the vehicle, and this is not seen in the NGF 6×/day group. This result requires further investigation to determine the biological significance, possibly a result of differential dependence of polymodal vs cold nociceptors on NGF.

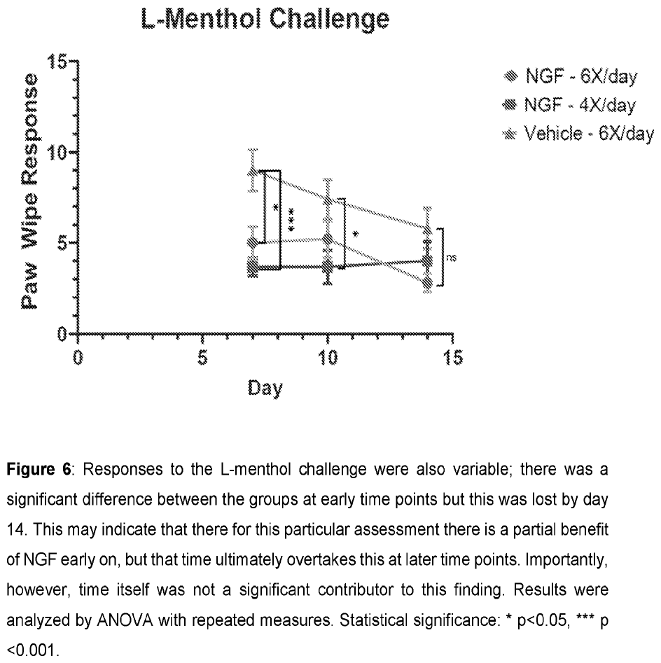

Response to L-Menthol: L-Menthol is capable of activating the transient receptor potential cation channel subfamily M member 8 (TRPM8), which has a major role in cold sensation. Thus, L-menthol was instilled to the ocular surface (10 μL) on the ligated side, within the immediate 30 second interval the number of paw wipes was counted. The number of paw wipes is used as a measure of pain, in that the greater the number of paw wipes performed by an animal then the greater the pain experienced by that animal. Results of this assessment are shown in FIG. 6. As indicated, there is a statistically significant difference between the NGF 4×/day group compared to the vehicle, and this is not seen in the NGF 6×/day group. Interestingly, these results do similarly mirror those found with cold saline alone. This result requires further investigation to determine the biological significance, possibly a result of differential dependence of polymodal vs cold nociceptors on NGF.

At day 14, trigeminal ganglia (TG) were excised for qRT-PCR analysis of neurotrophic factors and cytokines.

NGF treatment reduced levels of several neurotrophic factors in the TG compared to vehicle treatment (BDNF: 0.78 vs 1.00, p<0.05; NT-3: 0.25 vs 1.00, p<0.01; NT-4/5: 0.11 vs 1.00, p<0.05), but did not show increase in pro-inflammatory cytokines (IL-1b: 0.50 vs 1.00, IL-6: 0.92 vs 1.00, TNF-α: 0.69 vs 1.00; all p>0.05).

Summary of Clinical Outcomes: These data indicate that there appears to be a beneficial effect of NGF drops at both of the tested doses (6×/day and 4×/day). Importantly, there is no difference in corneal fluorescein staining between groups at any time point, and therefore, this model does not induce dry eye disease.

While there was a slight difference in corneal sensitivity as assessed by Cochet-Bonnet esthesiometry at early time points, this was lost by day 14. The most important take away from this assessment is that ligation does not destroy the ciliary nerves as corneal sensitivity is still intact. Importantly, there was a very significant improvement in response to the 5M saline challenge, the most useful of the pain outcomes. This effect was observed in both the 6× and 4× NGF treatment groups at all time points, with a complete return to baseline response by day 14 in the 6× group. The outcomes for cold saline and L-menthol are more variable, and may indicate either an early benefit of NGF, a difference in nociceptor dependence on NGF, or the result of improved surgical skill over time.

Taken together, the data from the retrospective clinical study of patients with NCP and the data from the murine model of NCP demonstrate that treatment with rhNGF results in an improved pain response for a subset of patients with NCP. The data further demonstrate the NGF treatment results in improvements in inflammation for a subset of patients with NCP.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

1-10. (canceled)

11. A method of treating neuropathic corneal pain (NCP) in a subject in need thereof, comprising administration of a composition comprising NGF.

12. The method according to claim 11, wherein the NGF is rhNGF.

13. The method according to claim 11, wherein the subject is a patient suffering from NCP who responds to treatment with a topical anesthetic and/or who has mild inflammation.

14. The method according to claim 13, wherein the topical anesthetic is proparacaine.

15. The method according to claim 11, wherein the subject has a Dendritiform Cell Density (cells/mm²) lower than 75.

16. The method according to claim 15, wherein the subject has a Dendritiform Cell Density (cells/mm²) lower than 70.

17. The method according to claim 11, wherein the subject is a patient suffering from peripheral or mixed NCP.

18. The method according to claim 11, wherein the subject is a patient who does not suffer from dry eye disease (DED) and/or from neurotrophic keratitis (NK).

19. The method according to claim 11, wherein NGF is administered topically to an ocular surface of the subject, in the form of a pharmaceutical composition for ophthalmic use.

20. The method according to claim 19, wherein NGF is present in the pharmaceutical composition at concentrations ranging from about 0.0001% to about 0.5% w/v.

21. The method according to claim 20, wherein NGF is present in the pharmaceutical composition at concentrations ranging from about 0.001% to about 0.1% w/v.

22. The method according to claim 21, wherein NGF is present in the pharmaceutical composition at a concentration of about 0.002% w/v.

23. The method according to claim 11, wherein the NGF is administered between 3 and 6 times a day for a period between 4 and 12 weeks.
